(19) United States
(12) Patent Application Publication
(10) Pub. No.: US 2018/0185258 A1
(43) Pub. Date: Jul. 5, 2018

(54) SKIN CARE COMPOSITIONS

(71) Applicant: Mayo Foundation for Medical Education and Research, Rochester, MN (US)

(72) Inventors: Brandon M. Kirsch, Neptune Beach, FL (US); Jason C. Sluzevich, Jacksonville, FL (US)

(73) Assignee: Mayo Foundation for Medical Education and Research, Rochester, MN (US)

(21) Appl. No.: 15/737,173

(22) PCT Filed: Jun. 16, 2016
(86) PCT No.: PCT/US2016/037908

§ 371 Date: Dec. 15, 2017

Related U.S. Application Data
(60) Provisional application No. 62/180,320, filed on Jun. 16, 2015.

(51) Int. Cl. A61K 8/49; A61K 8/362; A61K 8/27; A61Q 19/02; A61K 9/00; A61P 17/10; A61K 31/194; A61K 33/30; A61K 31/436; A61K 31/4436; A61Q 19/08

(52) U.S. Cl. CPC A61K 8/49; A61K 8/362; A61K 8/27; A61Q 19/02; A61K 9/0014; A61Q 19/08; A61K 31/194; A61K 33/30; A61K 31/436; A61K 31/4436; A61P 17/10

(57) ABSTRACT

This document provides methods and materials related to skin care. For example, skin care compositions containing azelaic acid, tacrolimus, tazarotene, and zinc oxide and methods for using such skin care compositions are provided.

### CROSS-REFERENCE TO RELATED APPLICATIONS

This application claims priority to U.S. Application Ser. No. 62/180,320, filed on Jun. 16, 2015. The disclosure of the prior application is considered part of the disclosure of this application, and is incorporated in its entirety into this application.

### BACKGROUND

### 1. Technical Field

This document relates to skin care and skin care compositions. For example, this document provides skin care compositions containing azelaic acid, zinc oxide, tacrolimus, and tazarotene that can be applied topically to the skin of a mammal (e.g., a human) for extended periods of time to lighten skin hyperpigmentation without atrophy, paradoxical hyperpigmentation, or other clinically limiting reactions.

### 2. Background Information

Skin lightening involves decreasing skin pigmentation caused by the deposition of melanin. Skin lightening is desirable since hyperpigmentation can be disfiguring and can cause emotional distress. Common examples of hyperpigmentation include melasma, post-inflammatory pigmentation (e.g., acne spots), and senile lentigo (e.g., sun, age, or liver spots).

### SUMMARY

This document provides methods and materials related to skin care. For example, this document provides skin care compositions and methods for using skin care compositions. In some cases, a skin care composition provided herein can contain azelaic acid, zinc oxide, tacrolimus, and tazarotene.

As described herein, a skin care composition (e.g., a skin care composition containing azelaic acid, zinc oxide, tacrolimus, and tazarotene) can be applied topically to the skin of a mammal (e.g., a human) for extended periods of time to treat a skin pigmentation condition. For example, a skin care composition (e.g., a skin care composition containing azelaic acid, zinc oxide, tacrolimus, and tazarotene) can be used topically and chronically to lighten skin hyperpigmentation without atrophy, paradoxical hyperpigmentation, or other clinically limiting reactions (e.g., inflammation, telangiectasias, or hypothalamic-pituitary adrenal axis suppression).

Having the ability to use the skin care compositions provided herein in a manner that allows a user (e.g., a human) to experience skin lightening by decreasing skin pigmentation caused by the deposition of melanin and/or preventing additional pigmentation above a natural coloration can allow the user to achieve and/or maintain a desired skin color result without atrophy, paradoxical hyperpigmentation, or other clinically limiting reactions.

In general, one aspect of this document features a skin care composition comprising, or consisting essentially of, from about 10 percent to about 30 percent, by weight, of azelaic acid, from about 5 percent to about 30 percent, by weight, of zinc oxide, from about 0.01 percent to about 3 percent, by weight, of tacrolimus, and from about 0.01 percent to about 0.2 percent, by weight, of tazarotene, wherein the skin care composition, when applied topically to a lentiginous macular hyperpigmentation of a human at least daily for at least 14 days, lightens the color of the lentiginous macular hyperpigmentation without atrophy or paradoxical hyperpigmentation. The composition can be a cream or lotion (or paste or self-dissolving medicinal clay). The composition can comprise from about 10 percent to about 20 percent, by weight, of the azelaic acid. The composition can comprise from about 5 percent to about 15 percent, by weight, of the zinc oxide. The composition can comprise from about 0.03 percent to about 0.10 percent, by weight, of the tacrolimus. The composition can comprise from about 0.02 percent to about 0.10 percent, by weight, of the tazarotene.

In another aspect, this document features a skin care composition comprising, or consisting essentially of, from about 10 percent to about 20 percent, by weight, of azelaic acid, from about 5 percent to about 15 percent, by weight, of zinc oxide, from about 0.03 percent to about 0.1 percent, by weight, of tacrolimus, and from about 0.02 percent to about 0.1 percent, by weight, of tazarotene. The composition can be a cream or lotion (or paste or self-dissolving medicinal clay).

In another aspect, this document features a method for lightening skin color. The method comprises, or consists essentially of, applying a skin care composition topically to an area of hyperpigmented mammalian skin under conditions wherein the color of the hyperpigmented mammalian skin is lightened. The skin care composition comprises, or consists essentially of, from about 10 percent to about 20 percent, by weight, of azelaic acid, from about 5 percent to about 15 percent, by weight, of zinc oxide, from about 0.03 percent to about 0.1 percent, by weight, of tacrolimus, and from about 0.02 percent to about 0.1 percent, by weight, of tazarotene. The hyperpigmented mammalian skin can be hyperpigmented human skin. The hyperpigmentation of the hyperpigmented mammalian skin can be an ephelide, café-au-lait macule, nevus spilus, senile lentigo, melasma, or post-inflammatory pigmentation. The color can be lightened without atrophy or paradoxical hyperpigmentation. The skin care composition can be applied topically to the area at least daily for at least 14 days. The skin care composition can be applied topically to the area at least daily for at least 60 days. The skin care composition can be applied topically to the area at least daily for at least 140 days. The skin care composition can be applied topically to the area at least daily for at least 140 days without causing atrophy or paradoxical hyperpigmentation.

In another aspect, this document features a method for treating acne vulgaris. The method comprises, or consists essentially of, applying a skin care composition topically to an area of mammalian skin under conditions wherein the skin affected by acne vulgaris is restored to normal. The skin care composition comprises, or consists essentially of, from about 10 percent to about 20 percent, by weight, of azelaic acid, from about 5 percent to about 15 percent, by weight, of zinc oxide, from about 0.03 percent to about 0.10 percent, by weight, of tacrolimus, and from about 0.02 percent to about 0.10 percent, by weight, of tazarotene. The skin can be human skin. The acne vulgaris of mammalian skin can be treated and returned to normal mammalian skin without atrophy. The skin care composition can be applied topically to the area at least daily for at least 5 days.

In another aspect, this document features a method for reducing signs of photo-induced or chronological aging of mammalian skin (e.g., fine lines, solar elastosis, skin roughening, and/or rhytids). The method comprises, or consists essentially of, applying a skin care composition topically to an area of mammalian skin under conditions wherein the skin affected by photo-induced or chronological aging is restored. The skin care composition comprises, or consists essentially of, from about 10 percent to about 20 percent, by weight, of azelaic acid, from about 5 percent to about 15 percent, by weight, of zinc oxide, from about 0.03 percent to about 0.10 percent, by weight, of tacrolimus, and from about 0.02 percent to about 0.10 percent, by weight, of tazarotene. The skin can be human skin. The photo-induced or chronological aging of mammalian skin can be treated and restored without atrophy. The skin care composition can be applied topically to the area at least about daily for at least about 30 days.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF THE DRAWINGS

FIG. 1 contains photographs of lentiginous macular hyperpigmentation present on the face of a human prior to treatment (A) and following treatment with a skin care composition containing azelaic acid (25%), zinc oxide (10%), tacrolimus (1.0%), and tazarotene (0.15%) twice daily for 14 days (B). Noticeable improvement was observed as only minimal pigmentation could be seen following treatment.

FIG. 2 contains photographs of a patient with inflammatory acneiform papules involving the distal mandible and chin before treatment (top left) and after treatment (bottom left). The inflammatory acneiform papules resolved after two weeks daily therapy (bottom left). Hypopigmented macules consistent with the subject's primary skin type against hyperpigmented background is shown before treatment (top right). Subtle expansion and lightening was observed after two weeks daily treatment (bottom right). Change also were evident in comparison of lateral cheeks (insets).

FIG. 3 contains photographs of a patient with infraorbital hyperpigmented coalescent macules before treatment (left). A gradual improvement after 30 days of daily treatment was observed (right).

### DETAILED DESCRIPTION

This document provides methods and materials related to skin care. For example, this document provides skin care compositions and methods for using skin care compositions. The term “skin care composition” as used herein refers to any product that is formulated for the topical application to skin (e.g., human skin) and that results in improving or maintaining a desirable skin color. For example, a skin care composition can result in skin lightening by decreasing skin pigmentation caused by the deposition of melanin and/or by preventing additional pigmentation above a natural coloration. In some cases, a skin care composition provided herein can achieve and/or maintain a desired skin color result without atrophy, paradoxical hyperpigmentation, or other clinically limiting reactions.

The skin care compositions provided herein can be applied to skin (e.g., a localized area of skin such as an area with ephelides, café-au-lait macules, nevus spilus, senile lentigo, melasma, post-inflammatory pigmentation, erythematous papules, pustules, comedones, fine lines, solar elastosis, skin roughening, and/or rhytids) in a manner that allows a user to experience a favorable outcome. In some cases, a skin care composition provided herein can be used to lighten hyperpigmentation from ephelides, café-au-lait macules, nevus spilus, senile lentigo, melasma, or post-inflammatory pigmentation. In some cases, a skin care composition provided herein can be applied to skin in a manner that allows a user to experience skin lightening without atrophy, paradoxical hyperpigmentation, or other clinically limiting reactions.

A skin care composition provided herein can be repeatedly applied to a mammal's skin (e.g., a human's skin) for an extended period of time (e.g., weeks, months, or years) without causing atrophy, paradoxical hyperpigmentation, or other clinically limiting reactions. For example, a skin care composition provided herein can be applied at least once a day (e.g., once or twice daily) or at least once a week (e.g., 1, 2, 3, 4, or 5 times a week) for at least 2, 3, 4, 5, 6, 7, or 8 weeks, for at least once daily for at least 1, 2, 3, 4, 5, 6, 7, or 8 months, or for at least once daily for at least 1, 2, 3, 4, or 5 years without causing atrophy, paradoxical hyperpigmentation, or other clinically limiting reactions.

In some cases, a skin care composition provided herein can be applied three times a day for two days and then two times a day until irritation (e.g., mild inflammation) can be seen. Depending on the degree of irritation, the skin care composition can be applied once or twice a day until depigmentation is complete (e.g., within about six to eight weeks or within about eight to twelve weeks). A maintenance treatment of about one application daily for from two to four days per week can be continued for one or more months (e.g., 1, 2, 3, 4, 5, 6, or more months). Treatment can be reinitiated as needed.

In some cases, a skin care composition provided herein can be used as a remedy for erythematous papules, pustules, and/or comedones to treat mammals (e.g., humans) with acne vulgaris. In some cases, a skin care composition provided herein can be used to reduce signs of photo-induced or chronological aging of mammalian skin (e.g., fine lines, solar elastosis, skin roughening, and/or rhytids).

In some cases, a skin care composition provided herein can contain azelaic acid, zinc oxide, tacrolimus, and tazarotene. A skin care composition provided herein can include any appropriate amount of azelaic acid. For example, a skin care composition provided herein can contain from about 10 percent to about 30 percent (e.g., from about 15 percent to about 25 percent, from about 15 percent to about 22 percent, or from about 19 percent to about 21 percent), by weight, of azelaic acid. In some cases, a skin care composition provided herein can contain about 20 percent of azelaic acid. In some cases, a skin care composition provided herein can contain kojic acid, aloesin, arbutin, resveratrol, oxyresveratrol, ellagic acid, or methyl gentisate in place of azelaic acid.

A skin care composition provided herein can include any appropriate amount of zinc oxide. For example, a skin care composition provided herein can contain from about 5 percent to about 30 percent (e.g., from about 5 percent to about 15 percent, from about 6 percent to about 14 percent, or from about 8 percent to about 12 percent), by weight, of zinc oxide. In some cases, a skin care composition provided herein can contain about 10 percent of zinc oxide. In some cases, a skin care composition provided herein can contain titanium dioxide, avobenzone, octyl methoxycinnamate (also known as octinoxate), oxybenzone, octocrylene, talc, or iron oxide in place of zinc oxide.

A skin care composition provided herein can include any appropriate amount of tacrolimus. For example, a skin care composition provided herein can contain from about 0.01 percent to about 3 percent (e.g., from about 0.01 percent to about 1 percent, from about 0.05 percent to about 0.8 percent, or from about 0.08 percent to about 0.15 percent), by weight, of tacrolimus. In some cases, a skin care composition provided herein can contain about 0.1 percent of tacrolimus. In some cases, a skin care composition provided herein can contain diclofenac sodium, oxicam, pyrazole, ibuprofen, diflunisal, or pimecrolimus in place of tacrolimus.

A skin care composition provided herein can include any appropriate amount of tazarotene. For example, a skin care composition provided herein can contain from about 0.01 percent to about 0.2 percent (e.g., from about 0.02 percent to about 0.2 percent, from about 0.05 percent to about 0.2 percent, or from about 0.08 percent to about 0.15 percent), by weight, of tazarotene. In some cases, a skin care composition provided herein can contain about 0.1 percent of tazarotene. In some cases, a skin care composition provided herein can contain about 0.075 percent of tazarotene. In some cases, a skin care composition provided herein can contain adapalene, all-trans retinoic acid, all-trans retinol, or bexarotene in place of tazarotene.

In some cases, a skin care composition provided herein can contain any appropriate combination of the ingredients. For example, a skin care composition provided herein can contain tazarotene, azelaic acid, one or more sunblock agents, and one or more anti-inflammatory agents. Examples of sunblock agents include, without limitation, titanium dioxide, zinc oxide, avobenzone, octyl methoxycinnamate (also known as octinoxate), oxybenzone, octocrylene, talc, or iron oxide. Examples of anti-inflammatory agents include, without limitation, diclofenac sodium, oxicam, pyrazole, ibuprofen, diflunisal, tacrolimus, or pimecrolimus.

A skin care composition provided herein can include any appropriate amount of a sunblock agent. For example, a skin care composition provided herein can contain from about 5 percent to about 30 percent (e.g., from about 5 percent to about 15 percent, from about 6 percent to about 14 percent, or from about 8 percent to about 12 percent), by weight, of a sunblock agent. In some cases, a skin care composition provided herein can contain about 10 percent a sunblock agent. In some cases, a skin care composition provided herein can have a sun protection factor of not less than 15.

A skin care composition provided herein can include any appropriate amount of an anti-inflammatory agent. For example, a skin care composition provided herein can contain from about 0.01 percent to about 10 percent (e.g., from about 0.03 percent to about 0.1 percent, from about 3 percent to about 5 percent, or from about 6 percent to about 10 percent), by weight, of an anti-inflammatory agent. In some cases, a skin care composition provided herein can contain about 0.1 percent of an anti-inflammatory agent.

In some cases, a skin care composition provided herein can include a fixed number of active ingredients. For example, a skin care composition provided herein can be formulated to have no more than three, four, five, six, seven, or eight active ingredients. In some cases, a skin care composition provided herein can be formulated to have four active ingredients (e.g., azelaic acid, zinc oxide, tacrolimus, and tazarotene) and no other active ingredients.

In some cases, a skin care composition provided herein can lack hydroquinone, hydroquinone monoethyl ether, hydroquinone monobenzyl ether, ammoniated mercury, mercurous chloride, bichloride of mercury and/or corticosteroids. For example, a skin care composition provided herein can be hydroquinone-free and/or corticosteroid-free.

A skin care composition provided herein can be in any appropriate form for topical application to skin (e.g., human skin). For example, a skin care composition provided herein can be in the form of a cream, gel, spray (e.g., an aerosol spray or non-aerosol spray), ointment, lotion, foam, solution, paste, or clay.

In some cases, a skin care composition provided herein can contain one or more optional classes of ingredients such pH adjusters, preservatives, solvents, viscosity increasing agents, excipients, emulsifiers, and emollients.

The final pH of the undiluted skin care composition provided herein can be between about 3 and about 8. To obtain such a final pH, the pH of the composition can be adjusted. A pH-adjusting agent can be used to adjust the pH. The pH adjustment can be accomplished with any of a wide variety of acids. Examples of acids that can be used include, without limitation, citric acid, acetic acid, benzoic acid, glycolic acid, lactic acid, malic acid, and sulfuric acid should the composition have a pH too high (e.g., greater than 8 before adjustment). Likewise, the pH adjustment can be accomplished with any of a wide variety of bases should the composition have a pH too low (e.g., less than 3 before adjustment). Examples of bases that can be used to lower the pH of these formulations can be potassium hydroxide, potassium carbonate, sodium carbonate, sodium hydroxide, ethanolamine, or triethanolamine.

In some cases, to preserve a skin care composition provided herein and prevent microbial growth, a preservative can be included in a skin care composition provided herein. Examples of preservatives that can be included in a skin care composition provided herein include, without limitation, butylated hydroxytoluene, benzoic acid, benzyl alcohol, butylparaben, propylparaben, methyparaben, DMDM hydantoin, potassium benzoate, methylisothiazolinone, methylchloroisothiazolinone, phenoxyethanol, quaterium-8, quaterium-14, quaterium-15, triclosan, zinc pyrithione, and zinc salicylate.

In some cases, one or more solvents can be included in a skin care composition provided herein as an optional ingredient. Examples of solvents that can be included in a skin care composition provided herein include, without limitation, butanediol, isoparaffin, cyclomethicone, ethoxyglycol, glycerin, mineral oil, polydimethlysiloxanes, propylene glycol, and propanediol.

In some cases, to help acquire a desired finished product thickness or viscosity, one or more viscosity modifiers can be included in a skin care composition provided herein. Examples of viscosity modifiers that can be included in a skin care composition provided herein include, without limitation, zinc oxide, ammonium xylene sulfonate, bentonite, calcium alginate, cocamide DEA, cocamide MEA, dextrin, hectorite, ethylcellulose, guar hydroxypropyltrimonium chloride, hydroxypropyl guar, hydrated silica, lauramide DEA, lauramide MEA, magnesium chloride, methylcellulose, pectin, polyethyleneglycol (PEGs), sodium chloride, sodium stearate, xanthan gum, and zea mays (corn starch).

In some cases, one or more excipients can be included in a skin care composition provided herein. Examples of excipients that can be included in a skin care composition provided herein include, without limitation, menthol, diglyceride, triglyceride, stabilizing agents, antioxidants, fragrances, and colorants.

In some cases, one or more emulsifiers and emollients can be included in a skin care composition provided herein. Examples of emulsifiers and emollients that can be included in a skin care composition provided herein include, without limitation, ceteareth-20, cetostearyl alcohol, diethylaminethyl stearate, glyceryl dilaurate, glyceryl monostearate, glyceryl stearate, PEG-100 stearate, octyldodecyl stearoyl stearate, polysorbate 80, quaternium-2β, stearyl alcohol, sodium PCA, dimethicone, cyclomethicone, propylene glycol, and polysiloxane derivatives. In some cases, an emulsifier or emollient can be present in a skin care composition provided herein at an amount from about 20 percent to about 80 percent, by weight.

The invention will be further described in the following examples, which do not limit the scope of the invention described in the claims.

### EXAMPLES

### Example 1

### Skin Care Composition

A skin care composition in the form of a paste was prepared by combining the ingredients listed in Table 1 such that the composition contained the indicated percentages by weight. This composition was prepared by adding azelaic acid flakes, tacrolimus monohydrate, and tazarotene powder to a mixture of propylene glycol in a Freedom Derma-D™ cream base, and then combined with a formulation of micronized zinc oxide.

TABLE 1
Example of skin care composition.

| Ingredient Name | % in Formulation |
|---|---|
| Azelaic acid | 25 |
| Tacrolimus | 1.0 |
| Tazarotene | 0.15 |
| Zinc oxide | 10 |

Analysis of this skin care composition revealed that it exhibits aesthetically pleasing characteristics such that it is smooth, non-comedogenic, and easy to blend without visible residue.

### Example 2

### Skin Care Composition

A skin care composition in the form of a self-dissolving clay was prepared by combining the ingredients listed in Table 2 such that the composition contained the indicated percentages by weight. This composition was prepared by adding azelaic acid flakes, tacrolimus monohydrate, tazarotene and zinc oxide (micronized) powder to a mixture of Vanicream™ and Vanicream Lite Lotion™ base.

TABLE 2
Example of skin care composition.

| Ingredient Name | % in Formulation |
|---|---|
| Azelaic acid | 20 |
| Tacrolimus | 0.1 |
| Tazarotene | 0.075 |
| Zinc oxide | 10 |

Analysis of this skin care composition revealed that it exhibits aesthetically pleasing characteristics such that it is smooth, non-comedogenic, and easy to blend without visible residue.

### Example 3

### Skin Care Composition

A skin care composition in the form of a cream is prepared by combining the ingredients listed in Table 3 such that the composition contains the indicated percentages by weight. This composition is prepared by adding azelaic acid flakes, tacrolimus monohydrate, tazarotene, and zinc oxide powder to a mixture of propylene glycol in a Freedom DermaD™ cream base.

TABLE 3
Example of skin care composition.

| Ingredient Name | % in Formulation |
|---|---|
| Azelaic acid | 20 |
| Tacrolimus | 0.03 |
| Tazarotene | 0.1 |
| Zinc oxide | 10 |

### Example 4

### Skin Care Composition

A skin care composition in the form of a cream is prepared by combining the ingredients listed in Table 4 such that the composition contains the indicated percentages by weight. This composition is prepared by adding azelaic acid flakes, tacrolimus monohydrate, tazarotene, and zinc oxide powder to a mixture of propylene glycol in a Freedom Derma-D™ cream base.

TABLE 4
Example of skin care composition.

| Ingredient Name | % in Formulation |
|---|---|
| Azelaic acid | 15 |
| Tacrolimus | 0.03 |
| Tazarotene | 0.1 |
| Zinc oxide | 15 |

### Example 5

### Skin Care Composition

A skin care composition in the form of a cream is prepared by combining the ingredients listed in Table 5 such that the composition contains the indicated percentages by weight. This composition is prepared by adding azelaic acid flakes, tacrolimus monohydrate, tazarotene, and zinc oxide powder to a mixture of propylene glycol in a Freedom Derma-D™ cream base.

TABLE 5
Example of skin care composition.

| Ingredient Name | % in Formulation |
|---|---|
| Azelaic acid | 15 |
| Tacrolimus | 0.03 |
| Tazarotene | 0.05 |
| Zinc oxide | 20 |

### Example 6

### Skin Care Composition

A skin care composition in the form of a cream is prepared by combining the ingredients listed in Table 6 such that the composition contains the indicated percentages by weight. This composition is prepared by adding azelaic acid flakes, tacrolimus monohydrate, tazarotene, and zinc oxide powder to a mixture of propylene glycol in a Freedom Derma-D™ cream base.

TABLE 6
Example of skin care composition.

| Ingredient Name | % in Formulation |
|---|---|
| Azelaic acid | 20 |
| Tacrolimus | 0.1 |
| Tazarotene | 0.05 |
| Zinc oxide | 15 |

### Example 7

### Skin Care Composition

A skin care composition in the form of a cream is prepared by combining the ingredients listed in Table 7 such that the composition contains the indicated percentages by weight. This composition is prepared by adding azelaic acid flakes, tacrolimus monohydrate, tazarotene, zinc oxide, and titanium dioxide powder to a Vanicream™ base.

TABLE 7
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |

### Example 8

### Skin Care Composition

A skin care composition in the form of a lotion is prepared by combining the ingredients listed in Table 8 such that the composition contains the indicated percentages by weight. This composition is prepared by combining azelaic acid flakes, tacrolimus monohydrate, tazarotene, zinc oxide, and titanium dioxide powder with menthol and rose oil and adding to a Vanicream Lite Lotion™ base.

TABLE 8
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |
| Menthol | 1 |
| Rose oil | 1 |

### Example 9

### Skin Care Composition

A skin care composition in the form of a cream is prepared by combining the ingredients listed in Table 9 such that the composition contains the indicated percentages by weight. This composition is prepared by combining azelaic acid flakes, tacrolimus monohydrate, tazarotene, zinc oxide, and titanium dioxide powder with menthol and rose oil and adding to a Vanicream™ base.

TABLE 9
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |
| Menthol | 1 |
| Rose oil | 1 |

### Example 10

### Skin Care Composition

A skin care composition in the form a cream is prepared by combining the ingredients listed in Table 10 such that the composition contains the indicated percentages by weight. This composition is prepared by combining azelaic acid flakes, tacrolimus monohydrate, tazarotene, and zinc oxide powder with menthol and resveratrol and blending with a mixture of propylene glycol in a Freedom Derma-D™ cream base.

TABLE 10
Example of skin care composition.

| Ingredient Name | % in Formulation |
|---|---|
| Azelaic acid | 20 |
| Tacrolimus | 0.05 |
| Tazarotene | 0.05 |
| Zinc oxide | 5 |
| Menthol | 1 |
| Resveratrol | 1 |

### Example 11

### Analysis of Skin Care Compositions

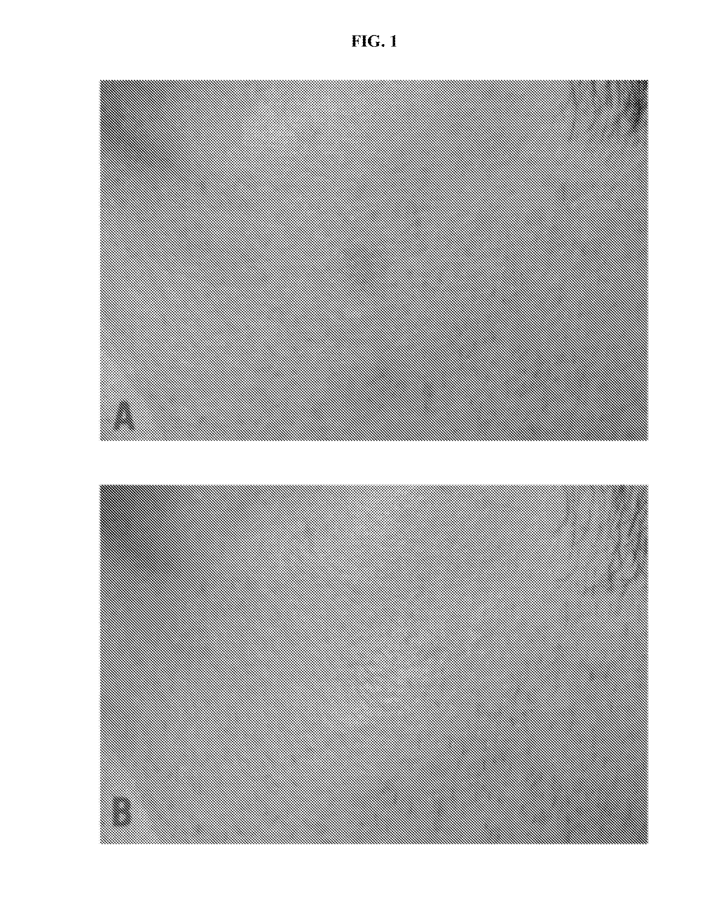

The skin care composition produced according to Example 1 was applied to a lentiginous macular hyperpigmentation present on the face of a human twice daily for 14 days. A photograph of the lentiginous macular hyperpigmentation was taken prior to treatment (FIG. 1A) and after 14 days of treatment (FIG. 1B). A noticeable improvement was observed as only minimal visible pigmentation could be seen following treatment.

### Example 12

### Skin Care Composition

A skin care composition in the form of a “self-dissolving medicinal clay” was prepared as described herein by combining the ingredients listed in Table 11 such that the composition contained the indicated percentages by weight. This composition was prepared by grinding, mixing, and milling azelaic acid flakes, tacrolimus monohydrate, and tazarotene into a combined Vanicream™ and Vanicream Lite Lotion™ base. Briefly, a ventilation hood was used until the powders were wet. Azelaic acid, tacrolimus, tazarotene, and zinc oxide powders as well as Vanicream™ and Vanicream Lite Lotion™ were weighed. In a mortar, the powders were mixed and ground until very fine. Small amounts of Vanicream™/Vanicream Lite Lotion™ were mixed into the powders until smooth. Geometrically, the remainder of the Vanicream™/Vanicream Lite Lotion™ was mixed in. The combination was missed well, and one ointment paper was used as necessary to ensure blending. The combination was then milled.

TABLE 11
Example of skin care composition.

| Ingredient Name | % in Formulation |
|---|---|
| Azelaic acid | 20 |
| Tacrolimus | 0.1 |
| Tazarotene | 0.075 |
| Zinc oxide | 10 |
| Vanicream ™ | 34 |
| Vanicream Lite Lotion ™ | 34 |

Analysis of this skin care composition revealed that it exhibits aesthetically pleasing characteristics such that it is smooth, non-comedogenic, and easy to blend without visible residue.

### Example 13

### Analysis of Skin Care Compositions

The skin care composition produced according to Example 12 was applied to seven humans to conduct a clinical trial into the treatment epidermal hyperpigmentation. Each human patient was directed to apply about 0.5 grams of the skin care composition topically to affected areas of skin once per day for 20 weeks. After 4 weeks, an initial assessment was completed, and the results were as follows.

The skin care composition was safe and well-tolerated. Patients experienced less than expected erythema, dryness, and scaling with application of the skin care composition. Without being limited to any particular mechanism of action, it is believed that the skin care composition exhibited better than expected tolerability due to its formulation as a medicinal clay with self-dissolving properties. This medicinal clay appeared to prevent excess application and overuse, thereby ensuring improved tolerability. In some cases, this medicinal clay can be used to deliver other topical medicines in a manner that prevents excess application.

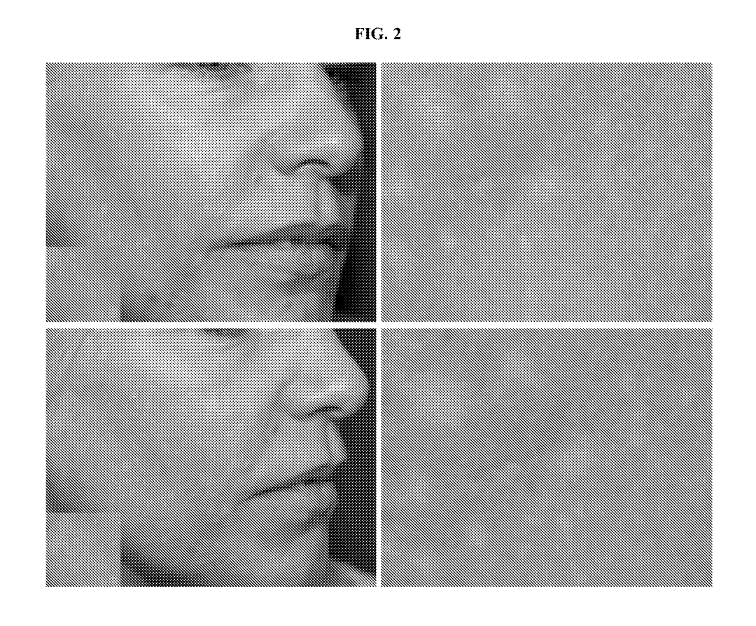

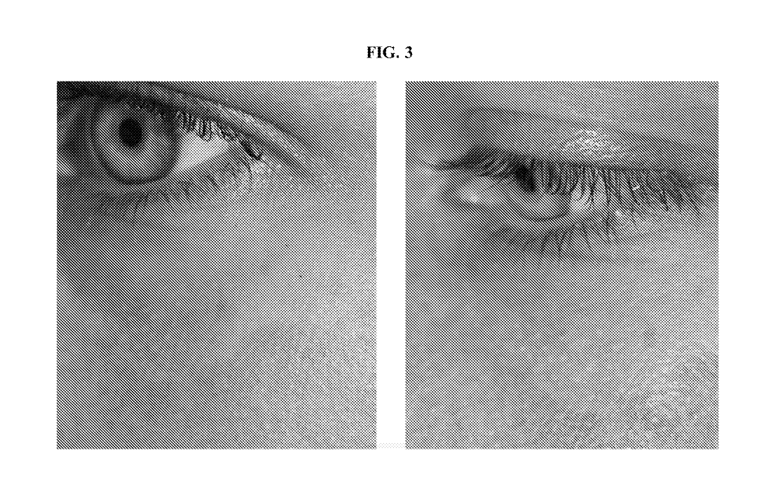

In addition, patients reported (a) a significant subjective improvement in softness and smoothness of skin, (b) improvement in the brightness and radiance of skin, (c) improvement in skin coloration, including lighter skin and more even skin tone, and (d) significant decreases in number and frequency of inflammatory acneiform papules. FIGS. 2 and 3 contain selected photographs before and after treatment. Patients also recommended the skin care composition to friends and family and expressed concern about their ability to procure the skin care composition following clinical trial conclusion.

These results demonstrate that the skin care compositions provided herein can be formulated as self-dissolving medicinal clays that are well-tolerated and efficacious for the treatment of epidermal hyperpigmentation. These results also demonstrate that the skin care compositions provided herein can have the additional benefit of enhancing skin brightness, radiance, smoothness, and softness, as well as reducing the number and frequency of inflammatory acneiform papules.

### Example 14

### Skin Care Composition

A skin care composition in the form of a cream is prepared by combining the ingredients listed in Table 12 such that the composition contains the indicated percentages by weight. This composition is prepared by adding azelaic acid flakes, tacrolimus monohydrate, and tazarotene powder to a mixture of propylene glycol in a Freedom Derma-D™ cream base, and then combining with a formulation of zinc oxide and titanium dioxide.

TABLE 12
Example of skin care composition.

| Ingredient Name | % in Formulation |
|---|---|
| Azelaic acid | 25 |
| Tacrolimus | 1.0 |
| Tazarotene | 0.15 |
| Zinc oxide | 10 |
| Titanium dioxide | 5.5 |

### Example 15

### Skin Care Composition

A skin care composition in the form of a cream is prepared by combining the ingredients listed in Table 13 such that the composition contains the indicated percentages by weight. This composition is prepared by adding azelaic acid flakes, tacrolimus monohydrate, tazarotene and zinc oxide powder to a mixture of propylene glycol in a Freedom DermaD™ cream base.

TABLE 13
Example of skin care composition.

| Ingredient Name | % in Formulation |
|---|---|
| Azelaic acid | 20 |
| Tacrolimus | 0.1 |
| Tazarotene | 0.1 |
| Zinc oxide | 10 |

### Example 16

### Skin Care Composition

A skin care composition in the form of a cream is prepared by combining the ingredients listed in Table 14 such that the composition contains the indicated percentages by weight. This composition is prepared by adding azelaic acid flakes, pimecrolimus, adapalene, and zinc oxide (micronized) powder to a Vanicream Lite Lotion™.

TABLE 14
Example of skin care composition.

| Ingredient Name | % in Formulation |
|---|---|
| Azelaic acid | 20 |
| Pimecrolimus | 1.0 |
| Adapalene | 0.1 |
| Zinc oxide | 10 |

### Example 17

### Skin Care Composition

A skin care composition in the form of a cream is prepared by combining the ingredients listed in Table 15 such that the composition contains the indicated percentages by weight. This composition is prepared by adding azelaic acid flakes, tacrolimus monohydrate, tretinoin and zinc oxide (micronized) powder to a mixture of Vanicream Lite Lotion™ base.

TABLE 15
Example of skin care composition.

| Ingredient Name | % in Formulation |
|---|---|
| Azelaic acid | 15 |
| Tacrolimus | 0.03 |
| Tretinoin | 0.1 |
| Zinc Oxide | 15 |

### Example 18

### Skin Care Composition

A skin care composition in the form of a cream is prepared by combining the ingredients listed in Table 16 such that the composition contains the indicated percentages by weight. This composition is prepared by adding azelaic acid flakes, tacrolimus monohydrate, tazarotene, and zinc oxide (micronized) powder to a mixture of Vanicream Lite Lotion™ base.

TABLE 16
Example of skin care composition.

| Ingredient Name | % in Formulation |
|---|---|
| Azelaic acid | 15 |
| Tacrolimus | 0.03 |
| Tazarotene | 0.05 |
| Zinc oxide | 20 |

### Example 19

### Skin Care Composition

A skin care composition in the form of a cream is prepared by combining the ingredients listed in Table 17 such that the composition contains the indicated percentages by weight. This composition is prepared by adding azelaic acid flakes, tacrolimus monohydrate, tazarotene, and zinc oxide (micronized) powder to a mixture of Vanicream Lite Lotion™ base.

TABLE 17
Example of skin care composition.

| Ingredient Name | % in Formulation |
|---|---|
| Azelaic acid | 20 |
| Tacrolimus | 0.1 |
| Tazarotene | 0.05 |
| Zinc oxide | 5 |

### Example 20

### Skin Care Composition

A skin care composition in the form of a self-dissolving clay is prepared by combining the ingredients listed in Table 18 such that the composition contains the indicated percentages by weight. This composition is prepared by adding azelaic acid flakes, tacrolimus monohydrate, tazarotene, zinc oxide (micronized), and titanium dioxide (micronized) powder to a Vanicream™ base.

TABLE 18
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |

### Example 21

### Skin Care Composition

A skin care composition in the form of a cream is prepared by combining the ingredients listed in Table 19 such that the composition contains the indicated percentages by weight. This composition is prepared by combining azelaic acid flakes, tacrolimus monohydrate, tazarotene, zinc oxide (micronized) powder, and titanium dioxide (micronized) powder with menthol and rose oil and adding to a Vanicream Lite Lotion™ base.

TABLE 19
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |
| Menthol | 1 |
| Rose oil | 1 |

### Example 22

### Skin Care Composition

A skin care composition in the form of a self-dissolving clay is prepared by combining the ingredients listed in Table 20 such that the composition contains the indicated percentages by weight. This composition is prepared by combining azelaic acid flakes, tacrolimus monohydrate, tazarotene, zinc oxide (micronized) powder, and titanium dioxide (micronized) powder with menthol and rose oil and adding to a Vanicream™ base.

TABLE 20
Example of skin care composition.

| Ingredient Name | % in Formulation |
|---|---|
| Azelaic acid | 20 |
| Tacrolimus | 0.05 |
| Tazarotene | 0.05 |
| Zinc oxide | 6 |
| Titanium dioxide | 3 |
| Menthol | 1 |
| Rose oil | 1 |

### Example 23

### Skin Care Composition

A skin care composition in the form a paste is prepared by combining the ingredients listed in Table 21 such that the composition contains the indicated percentages by weight. This composition is prepared by combining azelaic acid flakes, tacrolimus monohydrate, tazarotene, and zinc oxide (micronized) powder with menthol and resveratrol and blending with a mixture of propylene glycol in a Freedom Derma-D™ cream base.

TABLE 21
Example of skin care composition.

| Ingredient Name | % in Formulation |
|---|---|
| Azelaic acid | 20 |
| Tacrolimus | 0.05 |
| Tazarotene | 0.05 |
| Zinc oxide | 5 |
| Menthol | 1 |
| Resveratrol | 1 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A skin care composition comprising from about 10 percent to about 30 percent, by weight, of azelaic acid, from about 5 percent to about 30 percent, by weight, of zinc oxide, from about 0.01 percent to about 3 percent, by weight, of tacrolimus, and from about 0.01 percent to about 0.2 percent, by weight, of tazarotene, wherein said skin care composition, when applied topically to a lentiginous macular hyperpigmentation of a human at least daily for at least 14 days, lightens the color of said lentiginous macular hyperpigmentation without atrophy or paradoxical hyperpigmentation.

2. The composition of claim 1, wherein said composition is a cream or lotion.

3. The composition of claim 1, wherein said composition comprises from about 10 percent to about 20 percent, by weight, of said azelaic acid.

4. The composition of claim 1, wherein said composition comprises from about 5 percent to about 15 percent, by weight, of said zinc oxide.

5. The composition of claim 1, wherein said composition comprises from about 0.03 percent to about 0.10 percent, by weight, of said tacrolimus.

6. The composition of claim 1, wherein said composition comprises from about 0.02 percent to about 0.10 percent, by weight, of said tazarotene.

7. A skin care composition comprising from about 10 percent to about 20 percent, by weight, of azelaic acid, from about 5 percent to about 15 percent, by weight, of zinc oxide, from about 0.03 percent to about 0.1 percent, by weight, of tacrolimus, and from about 0.02 percent to about 0.1 percent, by weight, of tazarotene.

8. The composition of claim 7, wherein said composition is a cream or lotion.

9. A method for lightening skin color, wherein said method comprises applying a skin care composition topically to an area of hyperpigmented mammalian skin under conditions wherein the color of said hyperpigmented mammalian skin is lightened, wherein said skin care composition comprises from about 10 percent to about 20 percent, by weight, of azelaic acid, from about 5 percent to about 15 percent, by weight, of zinc oxide, from about 0.03 percent to about 0.1 percent, by weight, of tacrolimus, and from about 0.02 percent to about 0.1 percent, by weight, of tazarotene.

10. The method of claim 9, wherein said hyperpigmented mammalian skin is hyperpigmented human skin.

11. The method of claim 9, wherein the hyperpigmentation of said hyperpigmented mammalian skin is an ephelide, café-au-lait macule, nevus spilus, senile lentigo, melasma, or post-inflammatory pigmentation.

12. The method of claim 9, wherein said color is lightened without atrophy or paradoxical hyperpigmentation.

13. The method of claim 9, wherein said skin care composition is applied topically to said area at least daily for at least 14 days.

14-16. (canceled)

17. A method for treating acne vulgaris, wherein said method comprises applying a skin care composition topically to an area of mammalian skin under conditions wherein the skin affected by acne vulgaris is restored to normal, wherein said skin care composition comprises from about 10 percent to about 20 percent, by weight, of azelaic acid, from about 5 percent to about 15 percent, by weight, of zinc oxide, from about 0.03 percent to about 0.10 percent, by weight, of tacrolimus, and from about 0.02 percent to about 0.10 percent, by weight, of tazarotene.

18. The method of claim 17, wherein said skin is human skin.

19. The method of claim 17, wherein said acne vulgaris of mammalian skin is treated and returned to normal mammalian skin without atrophy.

20. The method of claim 17, wherein said skin care composition is applied topically to said area at least daily for at least 5 days.

21. A method for reducing signs of photo-induced or chronological aging of mammalian skin, wherein said method comprises applying a skin care composition topically to an area of mammalian skin under conditions wherein the skin affected by photo-induced or chronological aging is restored, wherein said skin care composition comprises from about 10 percent to about 20 percent, by weight, of azelaic acid, from about 5 percent to about 15 percent, by weight, of zinc oxide, from about 0.03 percent to about 0.10 percent, by weight, of tacrolimus, and from about 0.02 percent to about 0.10 percent, by weight, of tazarotene.

22. The method of claim 21, wherein said skin is human skin.

23. The method of claim 21, wherein said photo-induced or chronological aging of mammalian skin is treated and restored without atrophy.

24. The method of claim 21, wherein said skin care composition is applied topically to said area at least about daily for at least about 30 days.
